(19) United States
(12) Patent Application Publication
(10) Pub. No.: US 2015/0085992 A1
(43) Pub. Date: Mar. 26, 2015

(54) MOTORIZED DRIVE FOR MOBILE FLUOROSCOPY UNITS

(71) Applicant: John K. Grady, Ayer, MA (US)

(72) Inventor: John K. Grady, Ayer, MA (US)

(21) Appl. No.: 14/492,570

(22) Filed: Sep. 22, 2014

Related U.S. Application Data
(60) Provisional application No. 61/880,213, filed on Sep. 20, 2013.

(51) Int. Cl. A61B 6/00

(52) U.S. Cl. CPC A61B 6/4405; A61B 6/4441; A61B 6/487
USPC 378/198

(57) ABSTRACT

A mobile C-arm fluoroscopy unit comprising a self-contained radial coordinate or vector movement mechanism utilizing at least one motor-driven wheel, wherein the at least one motor-driven wheel is individually continuously steerable around a generally vertical steering axis.

### REFERENCE TO PENDING PRIOR PATENT APPLICATION

This patent application claims benefit of pending prior U.S. Provisional Patent Application Ser. No. 61/880,213, filed Sep. 20, 2013 by John K. Grady for MOTORIZED DRIVE FOR MOBILE FLUOROSCOPY UNITS (Attorney's Docket No. GRADY-1 PROV), which patent application is hereby incorporated herein by reference.

### FIELD OF THE INVENTION

The present invention relates generally to precision operator control and setting of the location of the center of a mobile X-ray unit beam, such as where X-ray fluoroscopy is used in the operating room on mobile or “portable” self-contained fluoro or C-arm units, particularly in the “Digital OR” or surgical navigation environment.

### BACKGROUND OF THE INVENTION

Prior approaches to X-ray positioning of mobile C-arms have required the use of a second carriage or “X” motion element mounted on the wheeled unit base to provide one axis of an X-Y system, the wheels on the floor providing the other or “Y” axis motion. The “Y” axis motion also provides travel mobility from room to room. Such a design is inherently limited in the amount of X-axis travel the second carriage provides as its cross-travel support bearings must overload if placed too close together; and such a carriage increases the width of the mobile unit, and can cause tipping instability, if the travel beyond the base footprint is made as large as it might be desired.

The prior art such as Galando (U.S. Pat. No. 6,374,937) and patents cited therein provide extensive detail of this approach, which can be summarized as providing a means to scan a patient in the operating room with a small field C-shaped X-ray viewer using X-Y drives. However, the units described in Galando (U.S. Pat. No. 6,374,937) consist of a mobile base often with swiveling or power driven castors or indexable wheels on the floor, always combined with an upper slide or carriage for the other (i.e., cross table) motion. The upper carriage motion is intended to be used at 90 degrees to the direction of travel of the wheels on the floor when the unit is in a position to be used on the patient, thus forming an X-Y coordinate system. The floor wheels provide the long travel (head to foot, or “Y” travel) and the second upper carriage provides the motion across the patient's width (or “X” travel).

See, for example, FIGS. 1 and 2, which show a mobile fluoroscopy unit 5 which generally comprises a mobile base 10 movable on powered wheels 15 so as to provide “Y” travel, and comprising a secondary carriage 20 movable on mobile base 10 so as to provide “X” travel, wherein a C-arm 25 is mounted to secondary carriage 20. Casters 30 provide support for mobile base 10. As a result of this construction, mobile base 10 may be moved on powered wheels 15 (and casters 30) so as to provide Y travel for the unit (and hence Y travel for C-arm 25) and secondary carriage 20 may be moved on mobile base 10 so as to provide X travel for C-arm 25.

The other prior art do not actively address steerable wheels at all, and depend on “grocery store-type” casters for steering.

With Galando (U.S. Pat. No. 6,374,937), the main travel wheels 15 of mobile base 10 may also be indexed to a “straight” position for moving mobile fluoroscopy unit 5 over substantial distances, e.g., throughout the hospital. Thus, with Galando (U.S. Pat. No. 6,374,937), the two indexed main travel wheel positions (and resulting travel motions) are “straight ahead” for moving the mobile fluoroscopy unit throughout the hospital, and 90 degrees from that when used with an X-ray stretcher or table. In other words, with Galando (U.S. Pat. No. 6,374,937), powered wheels 15 can be set for X travel while moving the unit into position, or for Y travel during positioning of C-arm 25 along the length of the patient (and with C-arm 25 then being set for X travel across the width of the patient using secondary carriage 20).

While this works reasonably well conceptually, there are serious limitations: the extent of cross-travel motion (i.e., the degree of X travel) is inherently limited to the width or length of the secondary carriage 20 (or traveling member) in the X direction, minus its support bearing spacing. This means a wide base is necessary (i.e., side-to-side, or 90 degrees from patient's long axis in use), but even if a wide base is provided, the overhanging mass of such a design will eventually cause the unit to fall over if the travel is made large.

For instance, in Galando (U.S. Pat. No. 6,374,937), at column 5, lines 31-43, the construction is described as a “preferred embodiment” and is in fact the only embodiment shown: the upper carriage (i.e., secondary carriage 20) construction is inherent to all the methods of use described in the patent.

Further, in Galando (U.S. Pat. No. 6,374,937), at column 6, lines 14-24, the invention is described as positioning the floor drive wheels into two positions 90 degrees from each other. In one position the wheels are pointed generally “straight ahead” for moving down a hospital corridor, while in the other position they are turned 90 degrees so that the unit travels along the long axis of the patient with the C-arm hanging to one side, over the patient's anatomy. Then, to position the X-ray beam across the patient, the second, limited travel upper carriage 20 is invoked in the X-direction. There is no other way to use the system described therein.

Again, in Galando (U.S. Pat. No. 6,374,937), at column 6, lines 56-58, the indexed wheels 15 are described as being pivoted from a “traveling position” to an “operating position”, clearly describing the intention of the vertical axis as indexing between two discrete positions.

In contrast, in the present invention discussed below, this vertical or steering axis is connected to a steering lever or steering input which is configured to direct the wheels in any arbitrary or vector direction, combined with a speed input control (preferably on the same lever or hand control). Using this input and mechanical arrangement, the entire upper “X” carriage motion described in Galando (and others) can be dispensed with or omitted; with the present invention, it is replaced with computer-operated servos vectoring the powered wheels in response to the operator's input.

Galando (U.S. Pat. No. 6,374,937) also calls out an X-Y joystick input device (see FIG. 2 of Galando) further indicative of the design intent of independent X-Y motions, even if actuated together.

Various known combinations of power-driven steerable castors, clutches and belts (such as are used in airport sweeping machines, ice rink conditioners, etc.) can accomplish this end goal of moving the X-ray C-arm centerline in a system of radial coordinates under the control a unique operator input device that communicates direction and velocity; an automotive steering wheel and throttle is a good example. However, the computer-assisted vector drive as described hereinbelow has not previously been applied to mobile X-ray units and requires unique solutions due to the overhanging C-arm mass.

Further, in Galando (U.S. Pat. No. 6,374,937), at column 9, lines 28-31, it is stated that “the apparatus of this invention, uses two distinct and separate chassis or carriages”. The invention discussed hereinbelow specifically disclaims use of two carriages for X-Y motion and is based on eliminating the X motion carriage entirely; instead, the present invention depends on a vector direction steering, and motion along that vector, to move the X-ray beam on the patient.

Further elaboration the X-Y approach of Galando (U.S. Pat. No. 6,374,937) is disclosed at: column 9, lines 32-97; column 10, lines 1-22; column 11, lines 43-51; column 12, lines 53-56; column 18, lines 39-41; and column 23, lines 17-23.

In Pejerski (U.S. Pat. No. 4,097,661), the intent is to steer a heavy device (an X-ray machine) by force sensors and a dual wheel drive, similar to a forklift truck. The system of the present invention described hereinbelow uses no force sensors and may only have a single driven wheel, such as a powered “ball” wheel, although two “motor-in-hub” wheels mounted (approximately at the center of gravity) to powered vertical steering axis pivots is the preferred method. The caster(s) used to stabilize the unit may also be actively steered, eliminating the need for offset axis or “tracking” casters.

### SUMMARY OF THE INVENTION

The present invention uses a precision “radial coordinate” drive concept on the main wheels (i.e., the powered wheels), which are used for all motions, including corridor travel and precision positioning at the patient, by selecting a “vector direction” (or steering direction), and speed, by using a suitable user interface, such as a lever which the operator points in a given direction and can “lift up” to start forward motion or “push down” for backup motion. The motion can be X, Y or any direction in between, by providing precision computer control of the wheel direction vector in a continuous manner. The position of the mobile fluoroscopy unit is known in a precise way and is intended to interact with a surgical navigation system in the operating room.

Combinations of wheel angles and speeds can be computer driven to accomplish the user goals.

Because of the various C-arm angling motions required for imaging at an angle, all mobile C-arm prior art using sliding collars on the C-arm will cause the beam tilt motions to interact in 3D space and are not generally isocentric. As a result, it becomes difficult to integrate a prior art “sliding collar” mobile C-arm into a surgical navigation system. For this reason, the present invention preferably uses the isocentric C-arm structure disclosed in U.S. Pat. No. 7,300,205 with such a navigation system, while supported on the novel wheeled system described herein.

It is also necessary to consider coordinated turns of the mobile fluoroscopy unit while being transported within the hospital. With the present invention, independent steering positioning servos for all the vectored wheels allows computer coordination of each wheel angle to the desired turn radius.

In one preferred form of the present invention, there is provided a mobile C-arm fluoroscopy unit comprising a self-contained radial coordinate or vector movement mechanism utilizing at least one motor-driven wheel, wherein the at least one motor-driven wheel is individually continuously steerable around a generally vertical steering axis.

In another preferred form of the present invention, there is provided a mobile C-arm fluoroscopy unit comprising:

a base;

at least one two powered wheels for moving the base relative to the floor; and

a C-arm mounted to the base;

wherein the each of the at least two powered wheels is (i) rotatable about a vertical axis, such that the wheel may be aimed in a given direction, and (ii) powered about a horizontal axis, such that the wheel may be driven in the given direction.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIGS. 1 and 2 are schematic views showing prior art mobile systems with indexable wheels;

FIGS. 3-5, 5A, 5B, 6 and 6A are schematic views showing the use of a vector drive or circular coordinates to position the X-ray beam on the patient, also incorporating straight-ahead drive and steering—many actual steering algorithms are known, such as allowing turning within its own footprint, once independence of direction and velocity are available at each vectored wheel; and

FIGS. 7-9 are schematic views showing a ¼-of-a-sphere cutout section on the side of the main body of the mobile fluoroscopy unit to allow full steering rotation in a vertical axis of a large main drive wheel.

### DETAILED DESCRIPTION OF THE PREFERRED EMBODIMENTS

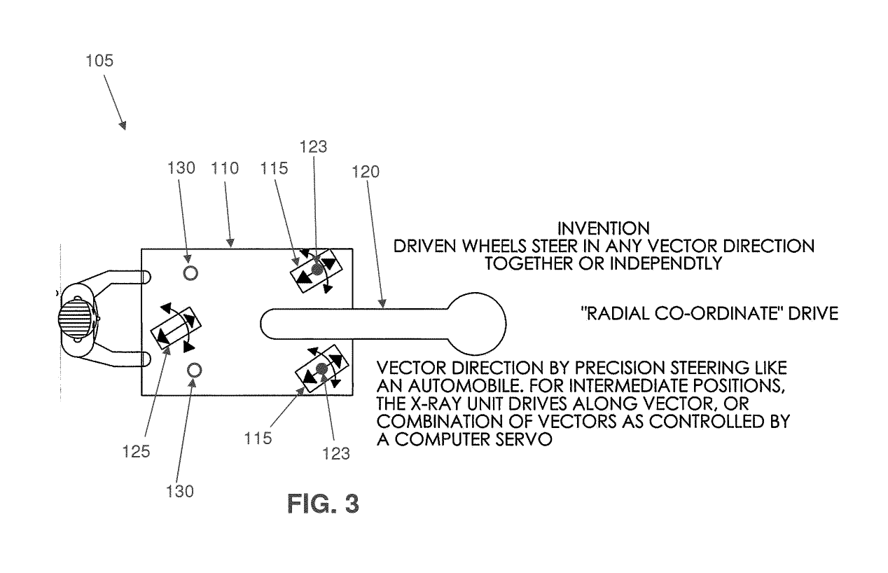

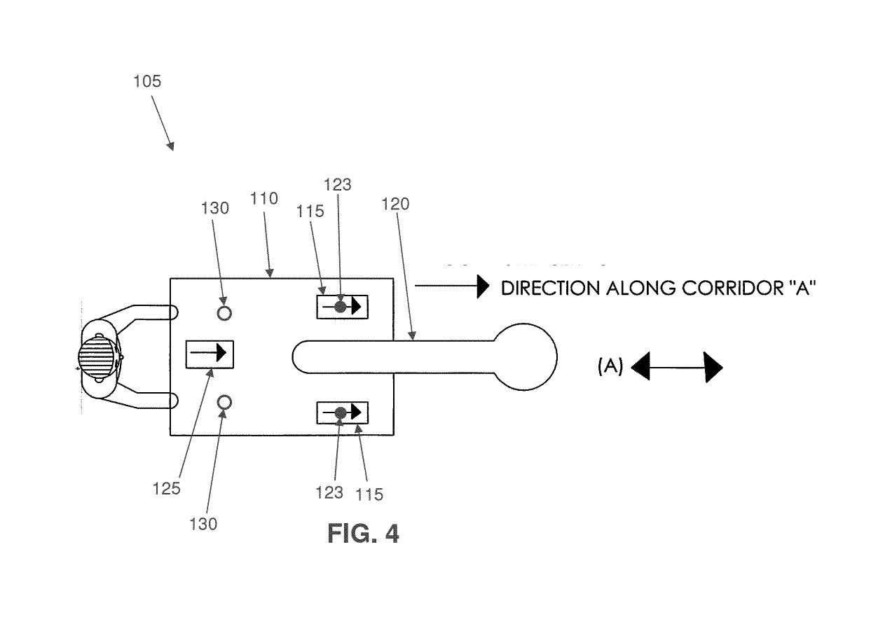

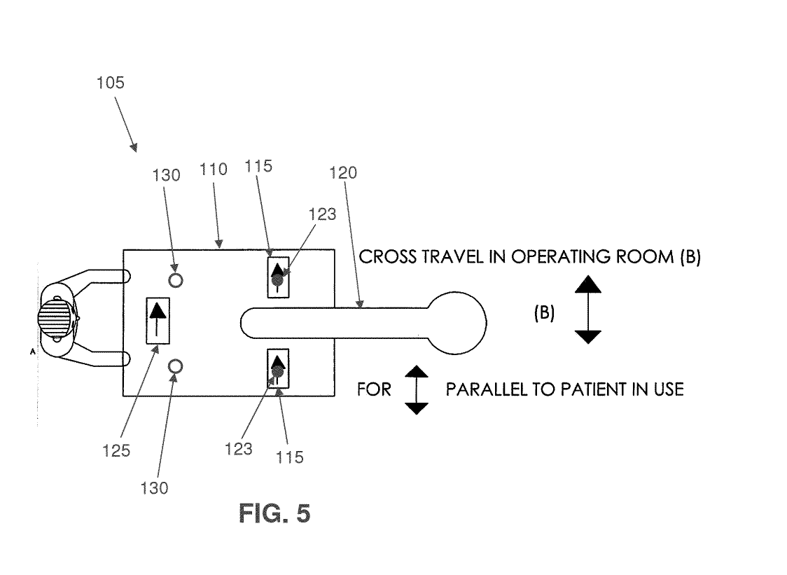

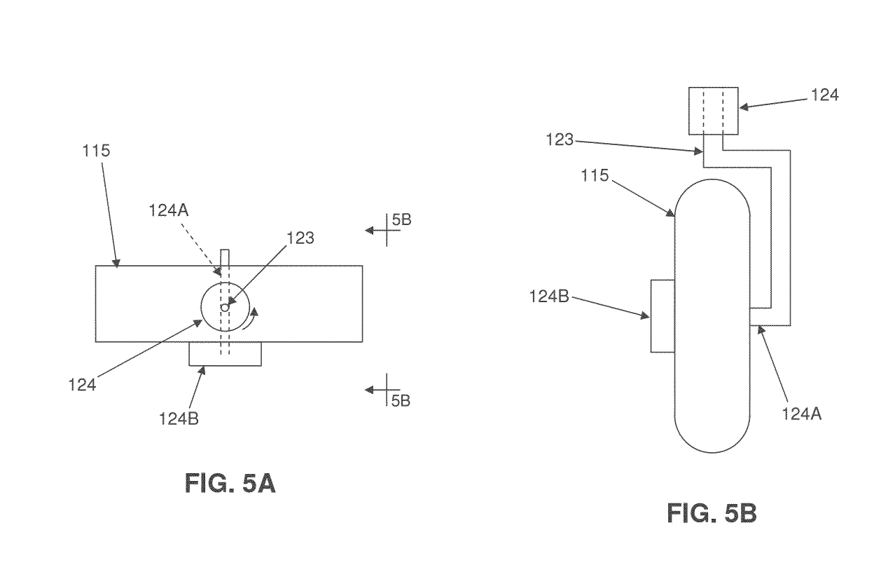

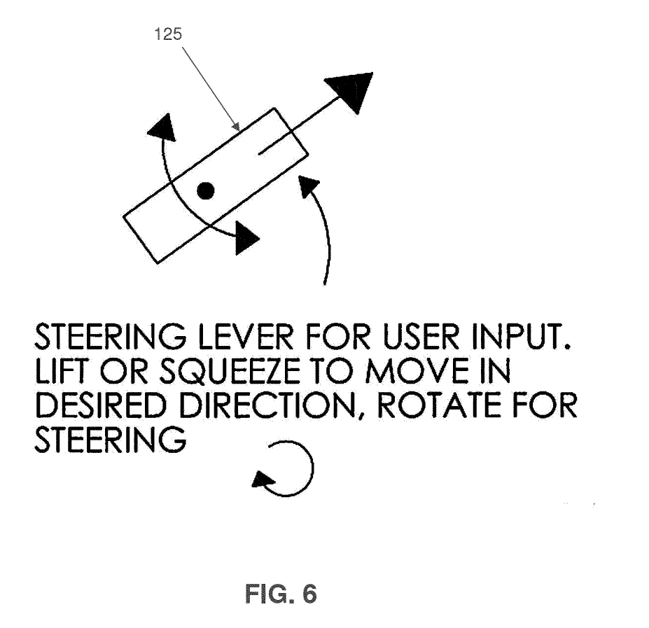

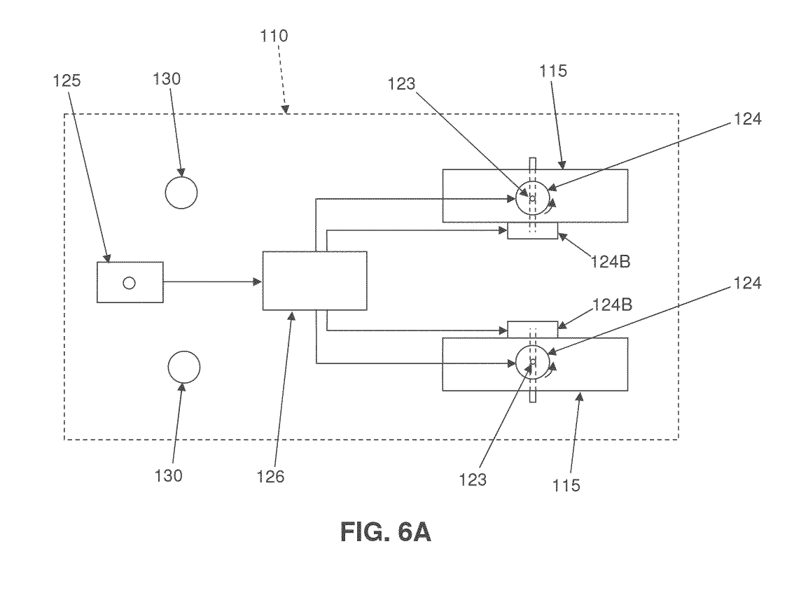

Looking now at FIGS. 3-6, the present invention comprises a mobile fluoroscopy unit 105 having a base 110, at least two powered wheels 115 for moving base 110 relative to the floor, and a C-arm 120 cantilevered relative to base 110. In accordance with the present invention, each of the at least two powered wheels 115 is configured to be rotated about a vertical axis 123 (e.g., via a vertical rotation mechanism 124) such that each of the at least two powered wheels 115 is directable in any direction. Each of the powered wheels 115 is configured to be rotated about a horizontal axis 124A (e.g., via a horizontal rotation mechanism 124B) such that each of the at least two powered wheels 115 can be driven in a given direction. A steering input 125 (e.g., a vertical or steering axis, such as a joystick or wheel) may be used to selectively aim powered wheels 115 in any arbitrary, or vector, direction. In one preferred form of the present invention, steering input 125 further comprises a speed input control on the same lever or hand control for controlling the speed movement of mobile fluoroscopy unit 105. By way of example but not limitation, a servo system 126 may be used to connect steering input 125 to vertical rotation mechanism 124, and horizontal rotation mechanism 124B, so that powered wheels 115 can be aimed, and driven, respectively, in a coordinated fashion, whereby to precisely position base 110, and hence C-arm 120, relative to a patient.

By virtue of this construction, the entire secondary carriage, which is necessary in the prior art to provide movement along the X-axis, can be eliminated. Instead, movement of mobile fluoroscopy unit 105 along the Y-axis and X-axis is provided by computer-operated servos which aim powered wheels 115 (i.e., by moving the wheels about vertical axis 123) and drive powered wheels 115 (i.e., by causing powered wheels 115 to move about a horizontal axle) and are attentive to the operator's input (e.g., via steering input 125).

If desired, casters 130 (free-wheeling or powered) may also be provided to help support base 110.

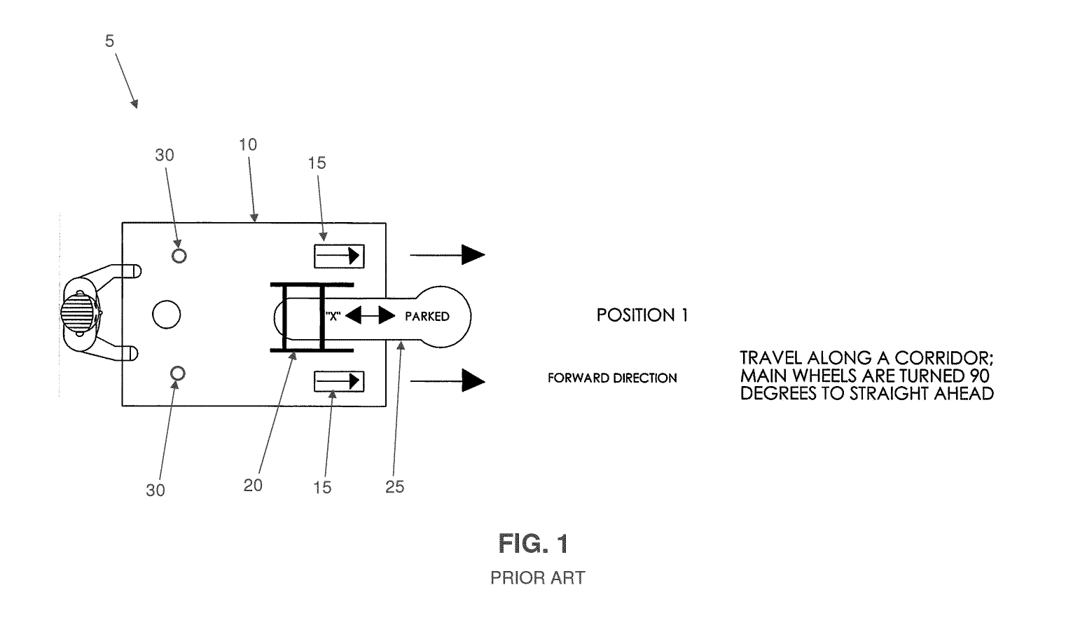

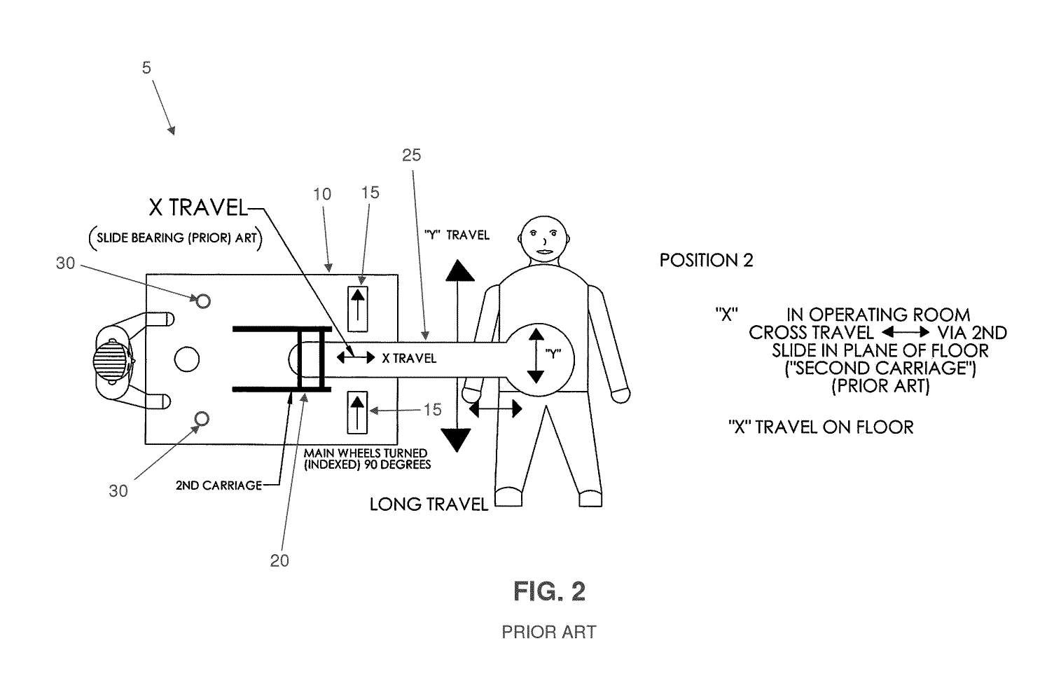

The present invention specifically eschews the prior art's use of a secondary carriage for X-axis movement (i.e., secondary carriage 20 of FIGS. 1 and 2) and instead relies solely on vector direction steering of powered wheels 115, and motion along that vector, to move the X-ray beam relative to the patient.

The present invention does not use force sensors, and, in one construction, utilizes only a single driven wheel 115, such as a powered “ball” wheel (although two “motor-in-hub” wheels 115, mounted approximately at the center of gravity to powered vertical steering axis pivots, is the preferred construction for the present invention). Caster(s) 130, which are used to stabilize base 110, may also be actively steered (in the same manner as powered wheels 115), thereby eliminating the need for offset axis or “tracking” casters.

The present invention comprises a precision “radial coordinate” drive concept on the main wheels 115, which are used for all motions, including corridor travel, by selecting a “vector direction” or steering direction, and a speed, using a suitable user interface (e.g., steering input 125). In use, an operator uses steering input 125 to point mobile fluoroscopy unit 105 in a desired direction and uses steering input 125 to move mobile fluoroscopy unit 105 (e.g., by “lifting up” on steering input 125 to start forward motion, or “pressing down” on steering input 125 to back up mobile fluoroscopy unit 105). Movement of mobile fluoroscopy unit 105 can be effected along the X-axis, along the Y-axis, or in any direction in between, by precision computer control of the wheel direction vector in a continuous way. The position of the mobile fluoroscopy unit 105 is known in a precise way and is intended to interact with a surgical navigation system in the operating room.

It will be appreciated that, if desired, combinations of wheel angles and speeds can be computer driven so as to effectively scan a patient using C-arm 125.

The various angling motions of C-arm 125, using sliding collars on the C-arm, causes the beam tilt motions to interact in 3D space and therefore the X-ray beam is not generally isocentric. Thus it becomes difficult to integrate the sliding collar mobile C-arms of the prior art into a surgical navigation system. The present invention uses an isocentric C-arm 125 (such as the isocentric C-arm disclosed in U.S. Pat. No. 7,300,205) in combination with a surgical navigation system, while the C-arm 125 is supported on the novel wheel system discussed above.

It should further be appreciated that, by providing independent steering positioning servos for all the powered wheels 115, the present invention allows computer coordination of each wheel angle to a desired turn radius, whereby to better navigate turning of mobile fluoroscopy unit 105 when moving mobile fluoroscopy unit 105 over long distances and/or tortured paths (e.g., such as hallways in a hospital).

The present invention uses vector drive and/or circular coordinates to position the X-ray beam on the patient, while also incorporating straight-ahead drive and steering. Many actual steering algorithms are known, such as steering algorithms which allow turning mobile fluoroscopy unit 105 within its own footprint, independent of direction and velocity, at each powered wheel 115.

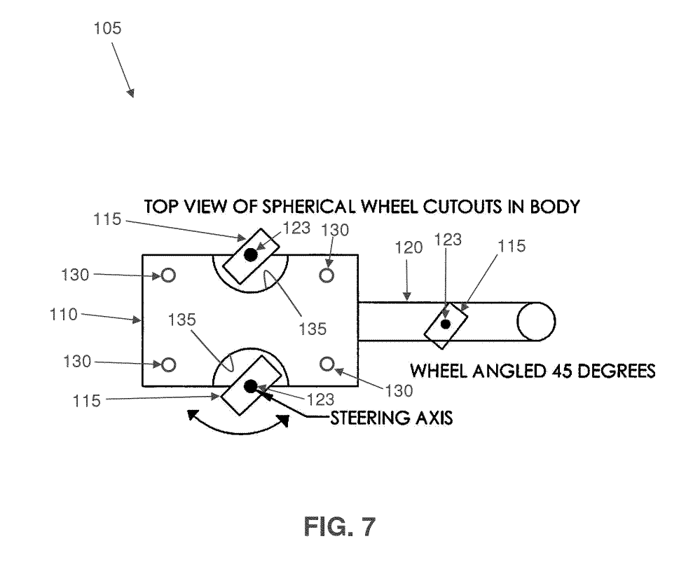

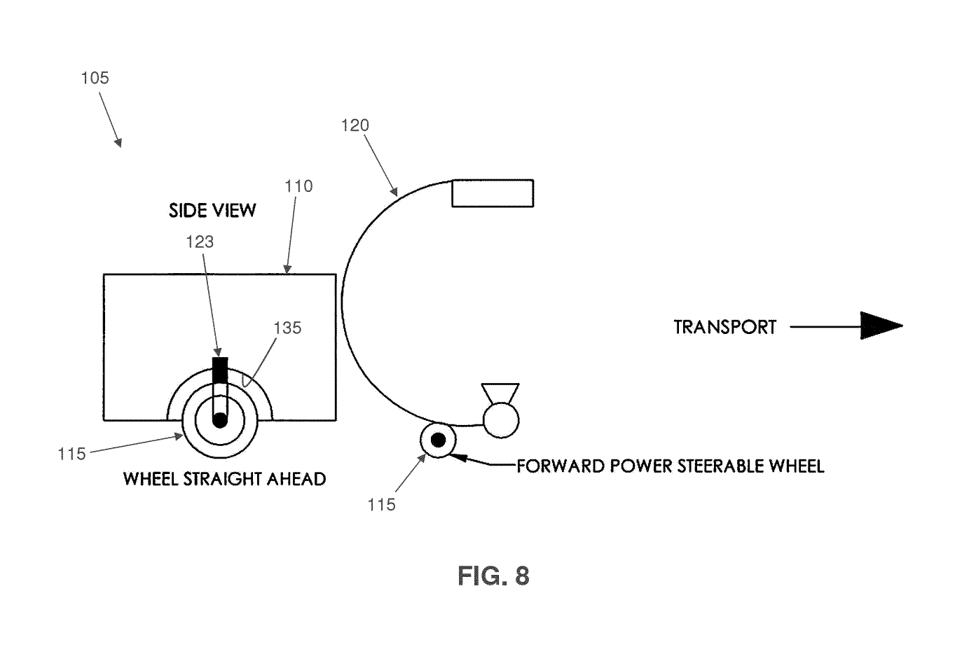

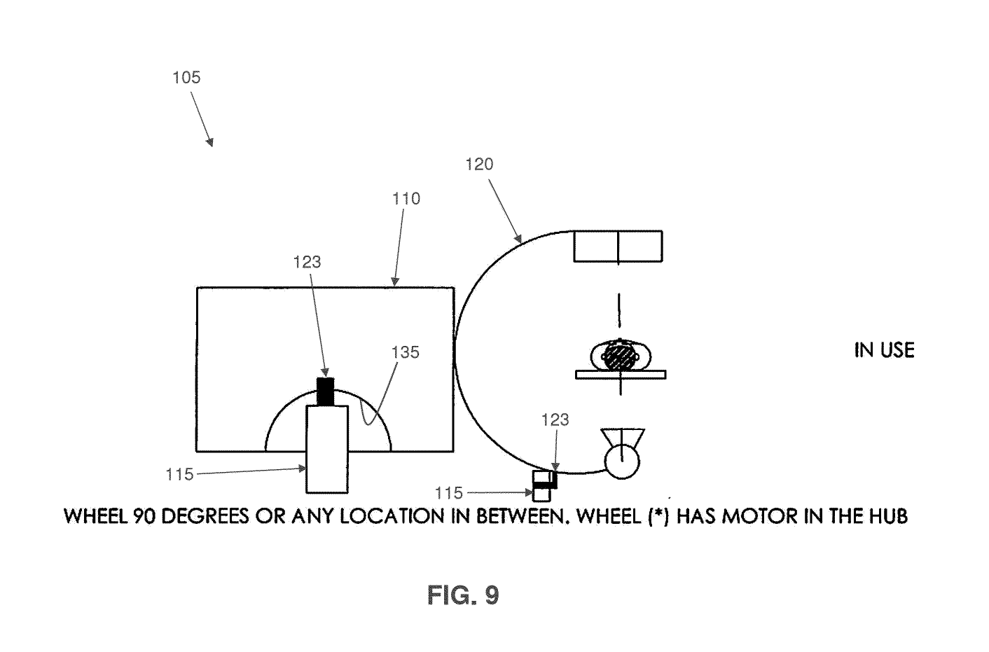

If desired, and looking now at FIGS. 7-9, wheels 115 may be provided in a ¼-of-a-sphere cutout section 135 on the side of the base 10 of mobile fluoroscopy unit 105 so as to allow full steering rotation in a vertical axis of a large main drive wheel 115. Furthermore, if desired, wheels 115 may be mounted on C-arm 120.

### MODIFICATIONS OF THE PREFERRED EMBODIMENTS

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A mobile C-arm fluoroscopy unit comprising a self-contained radial coordinate or vector movement mechanism utilizing at least one motor-driven wheel, wherein the at least one motor-driven wheel is individually continuously steerable around a generally vertical steering axis.

2. A mobile C-arm fluoroscopy unit according to claim 1 wherein the at least one motor-driven wheel is individually continuously steerable around a generally vertical steering axis by a user input steering vector, a power steering input, or a calculated angle.

3. A mobile C-arm fluoroscopy unit according to claim 1 wherein all movement of the unit relative to the floor is provided by the at least one motor-driven wheel rolling on the plane of the floor surface.

4. A mobile C-arm fluoroscopy unit according to claim 1 wherein the continuous steering action is accomplished by physically rotating the generally vertical steering axis by a precision computer servo drive.

5. A mobile C-arm fluoroscopy unit according to claim 1 wherein the continuous steering action is accomplished by physically rotating the generally vertical steering axis by a mechanical linkage.

6. A mobile C-arm fluoroscopy unit according to claim 1 wherein the vector disposition of the at least one wheel and the speed of rotation is measured and adjusted by a computer programmed so as to respond to an operator's input.

7. A mobile C-arm fluoroscopy unit according to claim 1 which interacts with an external coordinate reference system so as to position itself relative to the patient.

8. A mobile C-arm fluoroscopy unit according to claim 7 wherein the external coordinate reference system comprises a surgical navigation system.

9. A mobile C-arm fluoroscopy unit according to claim 8 wherein an image obtained by the mobile C-arm fluoroscopy unit is registered in the 3D space of the surgical navigation system.

10. A mobile C-arm fluoroscopy unit according to claim 1 configured to use the operating room table location and the mobile C-arm fluoroscopy unit location as inputs to the self-contained radial coordinate or vector movement mechanism.

11. A mobile C-arm fluoroscopy unit according to claim 10 configured to use an externally determined, and calibrated, external 3D grid to locate itself and the X-ray isocenter in 3D space.

12. A mobile C-arm fluoroscopy unit according to claim 1 comprising two large diameter main drive wheels each steerable in independent vertical axes, wherein a main body of the mobile C-arm fluoroscopy unit has a recess behind each wheel, shaped like a section of a sphere, to allow 360 degree steering rotation around the vertical steering axis.

13. A mobile C-arm fluoroscopy unit according to claim 1 comprising a plurality of motor-driven wheels, wherein all of the motor-driven wheels are independently angled or controlled by computer servo, so as to allow a coordinated turn while moving around any programmed virtual point or resulting turn radius.

14. A mobile C-arm fluoroscopy unit according to claim 1 wherein the C-arm is isocentric.

15. A mobile C-arm fluoroscopy unit comprising:
a base;
at least one two powered wheels for moving the base relative to the floor; and
a C-arm mounted to the base;
wherein the each of the at least two powered wheels is (i) rotatable about a vertical axis, such that the wheel may be aimed in a given direction, and (ii) powered about a horizontal axis, such that the wheel may be driven in the given direction.

16. A mobile C-arm fluoroscopy unit according to claim 15 further comprising an operator control system such that an operator may, for each of the at least two powered wheels, (i) control rotation of that wheel about a vertical axis, and (ii) control powering of that wheel about a horizontal axis.

17. A mobile C-arm fluoroscopy unit according to claim 16 wherein the operator control system allows coordinated operation of each of the at least two powered wheels.
